(19) United States
(12) Patent Application Publication
(10) Pub. No.: US 2025/0127446 A1
(43) Pub. Date: Apr. 24, 2025

(54) DEVICE, SYSTEM AND METHOD FOR ASSESSING WORKER FATIGUE

(71) Applicant: Makusafe Corp., West Des Moines, IA (US)

(72) Inventors: Mark Frederick, Cumming, IA (US); Gabriel Glynn, Ankeny, IA (US); Matthew McMullen, Omaha, NE (US); Nikhil Agarwal, Ankeny, IA (US)

(21) Appl. No.: 18/918,341

(22) Filed: Oct. 17, 2024

Related U.S. Application Data
(60) Provisional application No. 63/591,813, filed on Oct. 20, 2023.

(51) Int. Cl. A61B 5/22; A61B 5/00; A61B 5/11; G06Q 50/26; G16H 40/67

(52) U.S. Cl. CPC A61B 5/224; A61B 5/11; A61B 5/7267; A61B 5/7278; G06Q 50/265; G16H 40/67; A61B 2503/22

(57) ABSTRACT

In one or more arrangements, a system and method for assessing worker fatigue is presented. In one or more arrangements, the system includes a monitoring system and a wearable device configured to be worn by a worker during a work shift. The wearable device includes one or more sensors. The wearable device is configured to communicate motion data recorded by the wearable device the monitoring system. The monitoring system is configured to perform analytics on the motion data received from the wearable device to assess fatigue of the worker. In one or more arrangements, the monitoring system is configured to predict when a worker will become fatigued in the near future. In one or more arrangements, the monitoring system is configured to initiate one or more actions to mitigate the risk of accident or injury in response to determining a worker is or soon will be fatigued.

### CROSS REFERENCE TO RELATED APPLICATIONS

This application claims priority to U.S. Provisional Patent Application No. 63/591,813 filed Oct. 20, 2023 and titled “DEVICE, SYSTEM AND METHOD FOR ASSESSING WORKER FATIGUE”, which is hereby incorporated by reference herein in its entirety, including any figures, tables, or drawings or other information.

This application is related to U.S. patent application Ser. No. 18/176,748 filed Mar. 1, 2023 and titled DEVICE, SYSTEM AND METHOD FOR ASSESSING WORKER RISK; U.S. patent application Ser. No. 17/977,707 filed Oct. 31, 2022 and titled DEVICE, SYSTEM AND METHOD FOR HEALTH AND SAFETY MONITORING; U.S. Pub. No. 2021/0264764 filed May 6, 2021 and titled DEVICE, SYSTEM AND METHOD FOR HEALTH AND SAFETY MONITORING; U.S. Pat. No. 11,030,875, filed on Nov. 20, 2019 and titled SAFETY DEVICE, SYSTEM AND METHOD OF USE; U.S. Pat. No. 10,522,024 filed on Sep. 7, 2018 and titled SAFETY DEVICE, SYSTEM AND METHOD OF USE; and U.S. Pat. No. 10,096,230 filed on Jun. 6, 2017 and titled SAFETY DEVICE, SYSTEM AND METHOD OF USE, each of which is hereby incorporated by reference herein in its entirety, including any figures, tables, or drawings or other information.

### FIELD OF THE DISCLOSURE

This disclosure generally relates to monitoring systems. More specifically and without limitation, this disclosure relates to a monitoring system utilizing wearable devices to gather information indicative of work performed and/or work conditions.

### OVERVIEW OF THE DISCLOSURE

Injuries at work are tremendously costly for both the corporation as well as the injured worker. As an example, it is estimated that yearly workers' compensation claims exceed 100 billion dollars, with the average claim in the United State amounting to over 100,000 dollars.

Most, if not all of these work-related injuries are avoidable. In view of the personal cost to the injured worker and the financial cost to the employer, a great amount of energy and effort has been placed on avoiding workplace injuries. Many employers have implemented various systems to avoid accidents ranging from common sense solutions to sophisticated systems, from establishing safety teams and safety managers to hiring third-party safety auditors, and everything in-between. However, despite these many efforts, avoidable injuries continue to occur at an alarming pace.

To better inform and address workplace injuries, some current systems utilize wearable devices to gather data to evaluate movement, biometric data, environmental, or other data relevant to health and/or safety of workers. It is desired to be able to receive data from wearable devices to facilitate monitoring of workers throughout a work shift and facilitate early intervention when safety risks are detected and/or early response to accidents. For example, through careful observation and study it has been discovered that workers are more prone to mistakes and/or accidents when they become fatigued. However, current monitoring systems do not provide the capability to quantify or detect when workers are fatigued.

Therefore, there is a need in the art to provide a device, system, and method of use for collecting, reporting and analyzing information relating to workplace incidents, problems, potential concerns, work performed by workers and/or workplace conditions to better assess status of workers and risk posed to workers during a work shift.

Thus, it is a primary object of the disclosure to provide a wearable device, system and method of use that improves upon the state of the art.

Another object of the disclosure is to provide a wearable device, system and method of use that collects information about the work performed by workers, biometric data or workers, and/or workplace conditions.

Yet another object of the disclosure is to provide a wearable device, system and method of use that utilizes collected information to assess physicality exhibited by workers during a work shift and worker fatigue.

Another object of the disclosure is to provide a wearable device, system and method of use that utilizes collected information to assess physicality exhibited by workers during a work shift and identified when workers are fatigued based on the determined physicality.

Yet another object of the disclosure is to provide a wearable device, system and method of use that utilizes collected information to assess physicality exhibited by workers during a work shift and predict when workers will become fatigued based on the determined physicality.

Another object of the disclosure is to provide a wearable device, system and method of use that utilizes collected information to identify and/or predict when workers are fatigued and automatically perform a set of actions to mitigate risk of injury when fatigue is identified and/or predicted.

Yet another object of the disclosure is to provide a wearable device, system and method of use that analyzes data gathered to assess worker fatigue at multiple times throughout a work shift.

Another object of the disclosure is to provide a wearable device, system and method that more accurately assesses risk during a work shift.

Yet another object of the disclosure is to provide a wearable device, system and method of use that assesses gathered data indicative of work performed by workers and workplace conditions to facilitate assessment of safety risks faced by workers during a work shift.

Another object of the disclosure is to provide a wearable device, system and method of use that aggregates a great amount of information indicative of work performed by workers and workplace conditions to facilitate data analytics.

Yet another object of the disclosure is to provide a wearable device, system and method of use that is cost effective.

Another object of the disclosure is to provide a wearable device, system and method of use that is safe to use.

Yet another object of the disclosure is to provide a wearable device, system and method of use that is easy to use.

Another object of the disclosure is to provide a wearable device, system and method of use that is efficient to use.

Yet another object of the disclosure is to provide a wearable device, system and method of use that is durable.

Another object of the disclosure is to provide a wearable device, system and method of use that is robust.

Yet another object of the disclosure is to provide a wearable device, system and method of use that can be used with a wide variety of manufacturing facilities.

Another object of the disclosure is to provide a wearable device, system and method of use that is high quality.

Yet another object of the disclosure is to provide a wearable device, system and method of use that has a long useful life.

Another object of the disclosure is to provide a wearable device, system and method of use that can be used with a wide variety of occupations.

Yet another object of the disclosure is to provide a wearable device, system and method of use that provides high quality data.

Another object of the disclosure is to provide a wearable device, system and method of use that provides data and information that can be relied upon.

These and countless other objects, features, or advantages of the present disclosure will become apparent from the specification, figures, and claims.

### SUMMARY

In one or more arrangements, a system and method for assessing worker fatigue is presented. In one or more arrangements, the system includes a monitoring system and a wearable device configured to be worn by a worker during a work shift. The wearable device includes one or more sensors. The one or more sensors includes a motion sensor. The monitoring system is communicatively connected to the wearable device. The wearable device is configured to communicate window of motion data recorded by the motion sensor to the monitoring system. The monitoring system is configured to perform analytics on the motion data received from the wearable device to assess fatigue of the worker.

In one or more arrangements, the monitoring system is configured to assess fatigue of the worker by determining a power level exerted by the worker in each window of the motion data; sorting the determined power levels from highest to lowest to create a power curve; and assessing fatigue of the worker based on the power curve. In one or more arrangements, the monitoring system is configured to identify when the worker has become fatigued based on the power curve. In one or more arrangements, the monitoring system is configured to identify when the worker will become fatigued in the near future based on the power curve. In one or more arrangements, the monitoring system is configured to identify when the worker is becoming fatigued based on a comparison of the power curve to a baseline power curve for the worker. In one or more arrangements, the monitoring system is configured to identify when the worker is becoming fatigued by assessing the power curve using a machine learning algorithm that is trained to identify from the power curve when the worker is becoming fatigued.

In one or more arrangements, the monitoring system is configured to initiate one or more actions to mitigate the risk of accident or injury due to fatigue of the worker. In one or more arrangements, the monitoring system is communicatively connected to a status board configured to display workers currently working at a workstation and the monitoring system is configured to cause the status board to display a visual indicator warning when it is determined that the worker is becoming fatigued.

In one or more arrangements, in response to identifying that a worker is becoming fatigued, the monitoring system is configured to communicate a prompt for the worker to take a break. In one or more arrangements, in response to identifying that a worker is becoming fatigued, the monitoring system is configured to communicate a prompt for the worker to switch to a different work assignment. In one or more arrangements, in response to identifying that a worker is becoming fatigued, the monitoring system is configured to communicate a prompt for the worker to relocate to a more comfortable work location. In one or more arrangements, in response to identifying that a worker is becoming fatigued, the monitoring system is configured to prevent the workers from being permitted access to one or more restricted areas. In one or more arrangements, in response to identifying that a worker is becoming fatigued, the monitoring system is configured to prevent the workers from being permitted to operate one or more pieces of dangerous equipment.

### BRIEF DESCRIPTION OF THE DRAWINGS

FIG. 1 shows a system for monitoring workers for assessment of fatigue, in accordance with one or more arrangements.

FIG. 2 shows a block diagram of a wearable device for use with a system for monitoring workers for assessment of fatigue, in accordance with one or more arrangements.

FIG. 3 shows a flow chart of an example process for collecting and processing data by a wearable device, in accordance with one or more arrangements.

FIG. 4 shows a flow chart of an example process for collecting and processing data by a wearable device, in accordance with one or more arrangements.

FIG. 5 shows a flow chart of an example process for evaluating data collected by a wearable device to determine power exerted over time, in accordance with one or more arrangements.

FIG. 6 shows an example power curve in accordance with one or more arrangements.

FIG. 7 shows an example process for dynamic creation and/or update of a power curve during a work shift, in accordance with one or more arrangements.

FIG. 8A shows an example power curve updated at approximately 50% of the length of a work shift, in accordance with one or more arrangements; the power curve having a tail end of that is higher than the threshold, indicating that the worker has not yet become fatigued.

FIG. 8B shows the example power curve of FIG. 8A updated at approximately 66% of the length of the work shift; the power curve having a tail end of the power curve that remains higher than the threshold, indicating that the worker has not yet become fatigued; the power curve having predicted data points that are also above the threshold, indicating that the worker is not predicted to become fatigued in the near future.

FIG. 8C shows the example power curve of FIG. 8B updated at approximately 80% of the length of the work shift; the power curve having a tail end that remains higher than the threshold, indicating that worker has not yet become fatigued; the power curve having predicted data points that dip below the threshold, indicating that the worker is likely to become fatigued in the near future.

FIG. 9 shows an example process for dynamic creation and/or update of a power curve 170 and prediction of worker fatigue during a work shift, in accordance with one or more arrangements.

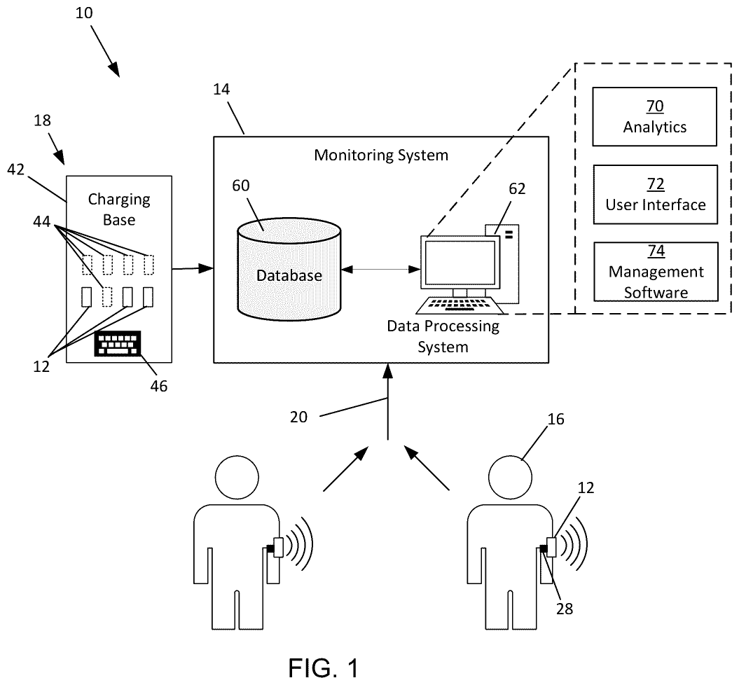

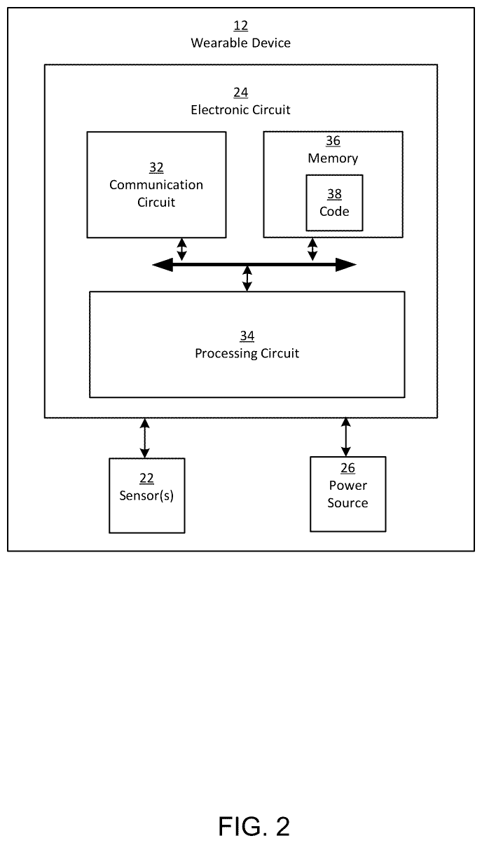

### DETAILED DESCRIPTION

In the following detailed description of the embodiments, reference is made to the accompanying drawings which form a part hereof, and in which is shown by way of illustration specific embodiments in which the disclosure may be practiced. The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure. It will be understood by those skilled in the art that various changes in form and details may be made without departing from the principles and scope of the invention. It is intended to cover various modifications and similar arrangements and procedures, and the scope of the appended claims therefore should be accorded the broadest interpretation so as to encompass all such modifications and similar arrangements and procedures. For instance, although aspects and features may be illustrated in or described with reference to certain figures or embodiments, it will be appreciated that features from one figure or embodiment may be combined with features of another figure or embodiment even though the combination is not explicitly shown or explicitly described as a combination. In the depicted embodiments, like reference numbers refer to like elements throughout the various drawings.

It should be understood that any advantages and/or improvements discussed herein may not be provided by various disclosed embodiments, or implementations thereof. The contemplated embodiments are not so limited and should not be interpreted as being restricted to embodiments which provide such advantages or improvements. Similarly, it should be understood that various embodiments may not address all or any objects of the disclosure or objects of the invention that may be described herein. The contemplated embodiments are not so limited and should not be interpreted as being restricted to embodiments which address such objects of the disclosure or invention. Furthermore, although some disclosed embodiments may be described relative to specific materials, embodiments are not limited to the specific materials or apparatuses but only to their specific characteristics and capabilities and other materials and apparatuses can be substituted as is well understood by those skilled in the art in view of the present disclosure.

It is to be understood that the terms such as “left, right, top, bottom, front, back, side, height, length, width, upper, lower, interior, exterior, inner, outer, and the like as may be used herein, merely describe points of reference and do not limit the present invention to any particular orientation or configuration.

As used herein, “and/or” includes all combinations of one or more of the associated listed items, such that “A and/or B” includes “A but not B,” “B but not A,” and “A as well as B,” unless it is clearly indicated that only a single item, subgroup of items, or all items are present. The use of “etc.” is defined as “et cetera” and indicates the inclusion of all other elements belonging to the same group of the preceding items, in any “and/or” combination(s).

As used herein, the singular forms “a,” “an,” and “the” are intended to include both the singular and plural forms, unless the language explicitly indicates otherwise. Indefinite articles like “a” and “an” introduce or refer to any modified term, both previously-introduced and not, while definite articles like “the” refer to a same previously-introduced term; as such, it is understood that “a” or “an” modify items that are permitted to be previously-introduced or new, while definite articles modify an item that is the same as immediately previously presented. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof, unless expressly indicated otherwise. For example, if an embodiment of a system is described at comprising an article, it is understood the system is not limited to a single instance of the article unless expressly indicated otherwise, even if elsewhere another embodiment of the system is described as comprising a plurality of articles.

It will be understood that when an element is referred to as being “connected,” “coupled,” “mated,” “attached,” “fixed,” etc. to another element, it can be directly connected to the other element, and/or intervening elements may be present. In contrast, when an element is referred to as being “directly connected,” “directly coupled,” “directly engaged” etc. to another element, there are no intervening elements present. Other words used to describe the relationship between elements should be interpreted in a like fashion (e.g., “between” versus “directly between,” “adjacent” versus “directly adjacent,” “engaged” versus “directly engaged,” etc.). Similarly, a term such as “operatively”, such as when used as “operatively connected” or “operatively engaged” is to be interpreted as connected or engaged, respectively, in any manner that facilitates operation, which may include being directly connected, indirectly connected, electronically connected, wirelessly connected or connected by any other manner, method or means that facilitates desired operation. Similarly, a term such as “communicatively connected” includes all variations of information exchange and routing between two electronic devices, including intermediary devices, networks, etc., connected wirelessly or not. Similarly, “connected” or other similar language particularly for electronic components is intended to mean connected by any means, either directly or indirectly, wired and/or wirelessly, such that electricity and/or information may be transmitted between the components.

It will be understood that, although the ordinal terms “first,” “second,” etc. may be used herein to describe various elements, these elements should not be limited to any order by these terms unless specifically stated as such. These terms are used only to distinguish one element from another; where there are “second” or higher ordinals, there merely must be a number of elements, without necessarily any difference or other relationship. For example, a first element could be termed a second element, and, similarly, a second element could be termed a first element, without departing from the scope of example embodiments or methods.

Similarly, the structures and operations discussed herein may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, to provide looping or other series of operations aside from single operations described below. It should be presumed that any embodiment or method having features and functionality described below, in any workable combination, falls within the scope of example embodiments.

As used herein, various disclosed embodiments may be primarily described in the context of gathering information for assessment of physicality and safety risk of workers. However, the embodiments are not so limited. It is appreciated that the embodiments may be adapted for use in other applications which may be improved by the disclosed structures, arrangements and/or methods. The system is merely shown and described as being used in the context of gathering information for assessment of physicality and worker risk for ease of description and as one of countless examples.

### System 10:

With reference to the figures, a system for collection of data indicative of worker activity and/or health and safety risks and identification and/or prediction of worker fatigue 10 (system 10) is presented. In one or more arrangements, system 10 includes a plurality of wearable devices 12 and a monitoring system 14 among other components.

### Wearable Devices 12:

Wearable devices 12 are formed of any suitable size, shape, and design and are configured to record motion and/or other data indicative of work performed by workers and/or safety risks encountered by workers during a work shift, such as environmental conditions as well as near misses. In one or more arrangements, recorded information may include, for example, motion of workers 16 (e.g., accelerometer and/or gyroscopic data), location of workers 16 during a work shift, proximity to high risk machinery, air quality, sound levels, data indicative of physicality of tasks performed by workers such as heart rate, temperature, perspiration level, number of steps, distance traveled, and/or other data acquired by sensors of wearable devices 12.

In one or more arrangements, system 10 may include wearable devices 12, charging base 18 and/or other components implemented as described in U.S. patent application Ser. No. 17/518,644 filed Nov. 4, 2021 and titled DEVICE, SYSTEM AND METHOD FOR ASSESSING WORKER RISK; U.S. patent application Ser. No. 17/977,707 filed Oct. 31, 2022 and titled DEVICE, SYSTEM AND METHOD FOR HEALTH AND SAFETY MONITORING; U.S. Pub. No. 2021/0264764 filed May 6, 2021 and titled DEVICE, SYSTEM AND METHOD FOR HEALTH AND SAFETY MONITORING; U.S. Pat. No. 11,030,875, filed on Nov. 20, 2019 and titled SAFETY DEVICE, SYSTEM AND METHOD OF USE; U.S. Pat. No. 10,522,024 filed on Sep. 7, 2018 and titled SAFETY DEVICE, SYSTEM AND METHOD OF USE; and U.S. Pat. No. 10,096,230 filed on Jun. 6, 2017 and titled SAFETY DEVICE, SYSTEM AND METHOD OF USE, each of which is hereby incorporated by reference herein in its entirety, including any figures, tables, or drawings or other information.

However, the embodiments are not so limited. Rather, it is contemplated that wearable devices 12 may be implemented using various other devices and/or arrangements configured to acquire sensor data and communicate recorded sensor data to monitoring system 14. In the arrangement shown, as one example, wearable devices 12 each include one or more sensors 22, an electronic circuit 24, and a power source 26 among other components.

### Sensors 22:

Sensors 22 are formed of any suitable size, shape, and design and are configured to sense various data metrics characterizing worker activity and/or environmental conditions surrounding the worker 16 while working. In one or more arrangements, wearable device 12 includes a plurality of sensors 22.

In one or more arrangements, wearable device 12 includes an accelerometer 22A. Accelerometer 22A is formed of any suitable size, shape, and design and is configured to detect acceleration and/or movement of the wearable device 12, such as when a worker 16 trips on something on the floor and almost falls, or when a worker 16 falls off of a ladder, is hit by a fork truck, or has another traumatic event. Accelerometer 22A may be formed of any acceleration detecting device such as a one axis accelerometer, a two-axis accelerometer, a three axis accelerometer or the like. Accelerometer 22A also allows for the detection of changes in acceleration, detection of changes in direction as well as elevated levels of acceleration.

In an alternative arrangement, or in addition to an accelerometer 22A, a gyroscope or gyro-sensor may be used to provide acceleration and/or movement information. Any form of a gyro is hereby contemplated for use, however, in one or more arrangements a three-axis MEMS-based gyroscope, such as that used in many portable electronic devices such as tablets, smartphones, and smartwatches are contemplated for use. These devices provide 3-axis acceleration sensing ability for X, Y, and Z movement, and gyroscopes for measuring the extent and rate of rotation in space (roll, pitch, and yaw).

In another arrangement, and/or in addition to an accelerometer 22A, a magnetometer may be used to provide acceleration and/or movement information. Any form of a magnetometer that senses information based on magnetic fields is hereby contemplated for use. In one or more arrangements, a magnetometer is used to provide absolute angular measurements relative to the Earth's magnetic field. In one or more arrangements, an accelerometer, gyro and/or magnetometer are incorporated into a single component or a group of components that work in corresponding relation to one another to provide up to nine axes of sensing in a single integrated circuit providing inexpensive and widely available motion sensing.

In one or more arrangements, wearable device 12 includes a temperature sensor 22B. Temperature sensor 22B is formed of any suitable size, shape, and design and is configured to detect the temperature of the environment surrounding the worker 16. The same and/or an additional temperature sensor 22B may be configured to detect the temperature of the worker 16 themselves. In one or more arrangements, temperature sensor 22B is a thermometer. Temperature sensor 22B allows for the detection of high or low temperatures as well as abrupt changes in temperature. Temperature sensor 22B also allows for the detection of when a temperature threshold is approached or exceeded. In one or more arrangements, wearable device 12 includes a humidity sensor 22C. Humidity sensor 22C is formed of any suitable size, shape, and design and is configured to detect the humidity of the environment surrounding the worker 16. The same and/or an additional humidity sensor 22C may be configured to detect the humidity level, moisture level or perspiration level of the worker 16 themselves. Humidity sensor 22C allows for the detection of high or low levels of humidity as well as abrupt changes in humidity. Humidity sensor 22C also allows for the detection of when a humidity threshold is approached or exceeded. In one or more arrangements, wearable device 12 includes a light sensor 22D. Light sensor 22D is formed of any suitable size, shape, and design and is configured to detect the light levels of the environment surrounding the worker 16. Light sensor 22D allows for the detection of high or low levels of light as well as abrupt changes in light levels. Light sensor 22D also allows for the detection of when a light threshold is approached or exceeded. In one or more arrangements, light sensor 22D is operably connected to and/or accessible by a light pipe (not shown). Light pipe is any device that facilitates the collection and transmission of light from the environment surrounding the worker 16. In one or more arrangements, light pipe is a clear, transparent, or translucent material that extends from the exterior of the wearable device 12 to the light sensor 22D and therefore covers and protects light sensor 22D while enabling the sensing of light conditions.

In one or more arrangements, wearable device 12 includes an air quality sensor 22E. Air quality sensor 22E is formed of any suitable size, shape, and design and is configured to detect the air quality of the environment surrounding the worker 16, the particulate matter in the air of the environment surrounding the worker 16, the contaminant levels in the air of the environment surrounding the worker 16, or any particular contaminant level in the air surrounding the worker 16 (such as ammonia, chlorine, or any other chemical, compound or contaminant). Air quality sensor 22E allows for the detection of high contaminant levels in the air as well as abrupt changes in air quality. Air quality sensor 22E also allows for the detection of when an air quality threshold is approached or exceeded.

In one or more arrangements, air quality sensor 22E is a total volatile organic compound sensor, also known as a TVOC sensor. Volatile organic compounds (or VOCs) are organic chemicals that have a high vapor pressure at ordinary room temperature. VOCs are numerous, varied, and ubiquitous. They include both human-made and naturally occurring chemical compounds. Most scents or odors are of VOCs. In this arrangement, air quality sensor 22 is configured to detect VOCs. Also, in one or more arrangements, air quality sensor 22E is accessible through one or more openings in wearable device 12 that provide unfettered access and airflow for sensing by air quality sensor 22E.

In one or more arrangements, wearable device 12 includes a carbon monoxide (CO) sensor 22F. CO sensor 22F is formed of any suitable size, shape, and design and is configured to detect CO levels of the environment surrounding the worker 16. CO sensor 22F allows for the detection of high CO levels in the air as well as abrupt changes in CO levels. CO sensor 22F also allows for the detection of when a CO threshold is approached or exceeded. Of course, sensor 22F, or additional sensors 22, may be used to sense other gasses in the air around the worker 16, such as carbon dioxide, ozone, or any other gas or other content of the air around the worker 16. Also, in one or more arrangements, sensor 22F is accessible through one or more openings in wearable device 12 that provide unfettered access and airflow for sensing by sensor 22F.

In one or more arrangements, wearable device 12 includes a position sensor 22G. Position sensor 22G is formed of any suitable size, shape, and design and is configured to detect the position of the worker 16 within the manufacturing facility. Notably, the term manufacturing facility is to be construed in a broad manner and may include being within one or a plurality of buildings. However, the manufacturing facility may include being outside and unconstrained by the boundaries of a building or any particular grounds. Position sensor 22G allows for the detection of movement of the worker 16 within the manufacturing facility, the speed of movement of the worker 16 within the manufacturing facility, the tracking of the position of the worker 16 within the manufacturing facility, among any other speed, location, direction, inertia, acceleration or position information. This position information can be aggregated over the course of the worker's shift to determine the amount of distance traveled by the worker 16, the average speed, the mean speed, the highest speed, or any other information. In addition, this position information can be aggregated to determine the areas where the worker 16 concentrated their time. In addition, this position information can be correlated with the information detected by the other sensors to determine the concentration of certain environmental factors in different areas of the manufacturing facility. Position sensor 22G may be a GPS device, a wireless device (e.g., Wi-Fi and/or RFID) configured to detect presence of nearby wearable devices, a wireless device that utilizes trilateration from known points, or any other device that detects the position of wearable device 12 and the worker 16.

Wearable device 12 may also include any other sensors 22. For example, in one or more arrangements, wearable device 12 includes one or more sensor 22 that tracks biometric data of the worker 16 including but not limited to, for example, heart rate, blood pressure, blood oxygen levels, blood alcohol levels, blood glucose sensor, respiratory rate, galvanic skin response, bioelectrical impedance, brain waves, and/or combinations thereof.

In one or more arrangements, wearable device 12 includes a sound sensor 22H. Sound sensor 22H is formed of any suitable size, shape, and design and is configured to detect the volume level and/or frequency of sound surrounding the worker 16. In one or more arrangements, sound sensor 22H is a microphone that is accessible through one or more openings in wearable device 12 that provide unfettered access for the sound to reach the microphone. Sound sensor 22H allows for the detection of elevated sounds, abrupt spikes in sounds, loud noises, irritating or distracting frequencies or the like. Sound sensor 22H also allows for the detection of when a volume threshold is approached or exceeded.

During operation, sensors 22 detect environmental conditions, such as sound, temperature, humidity, light, air quality, CO levels, TVOC levels, particulate levels, position and acceleration information, direction information, speed information and the like respectively.

### Electronic Circuit 24:

Electronic circuit 24 is formed of any suitable size, shape, design, technology, and in any arrangement and is configured to facilitate retrieval, processing, and/or communication of data from sensor(s) 22 of wearable device 12 to monitoring system 14. In the arrangement shown, as one example, electronic circuit 24 includes a communication circuit 32, a processing circuit 34, and a memory 36 having software code 38 or instructions that facilitates the operation of wearable device 12.

In one or more arrangements, electronic circuit 24 includes a communication circuit 32. Communication circuit 32 is formed of any suitable size, shape, design, technology, and in any arrangement and is configured to facilitate communication with monitoring system 14. In one or more arrangements, as one example, communication circuit 32 includes a transmitter (for one-way communication) or transceiver (for two-way communication). In some various arrangements, communication circuit 32 may be configured to communicate with monitoring system 14 and/or various components of system 10 using various wired and/or wireless communication technologies and protocols over various networks and/or mediums including but not limited to, for example, IsoBUS, Serial Data Interface 12 (SDI-12), UART, Serial Peripheral Interface, PCI/PCIe, Serial ATA, ARM Advanced Microcontroller Bus Architecture (AMBA), USB, Firewire, RFID, Near Field Communication (NFC), infrared and optical communication, 802.3/Ethernet, 802.11/WIFI, Wi-Max, Bluetooth, Bluetooth low energy, UltraWideband (UWB), 802.15.4/ZigBee, ZWave, GSM/EDGE, UMTS/HSPA+/HSDPA, CDMA, LTE, 4G, 5G, FM/VHF/UHF networks, and/or any other communication protocol, technology or network.

In some various arrangements, electronic circuit 24 and/or communication circuit 32 may be configured to communicate data from sensors 22 to monitoring system 14 (or other device) continuously, periodically, according to a schedule, when prompted by monitoring system 14 (or other device), when wearable device is checked in and connected to charging base 18, and/or in response to any other stimuli, command, or event.

Processing circuit 34 may be any computing device that receives and processes information and outputs commands, for example, according to software code 38 stored in memory 36. For instance, in some various arrangements, processing circuit 34 may be discreet logic circuits or programmable logic circuits configured for implementing these operations/activities, as shown in the figures and/or described in the specification. In certain arrangements, such a programmable circuit may include one or more programmable integrated circuits (e.g., field programmable gate arrays and/or programmable ICs). Additionally or alternatively, such a programmable circuit may include one or more processing circuits (e.g., a computer, microcontroller, system-on-chip, smart phone, server, and/or cloud computing resources). For instance, computer processing circuits may be programmed to execute a set (or sets) of software code stored in and accessible from memory 36. Memory 36 may be any form of information storage such as flash memory, ram memory, dram memory, a hard drive, or any other form of memory.

In one or more arrangements, processing circuit 34 and memory 36 may be formed of a single combined unit. Alternatively, processing circuit 34 and memory 36 may be formed of separate but electrically connected components. Alternatively, processing circuit 34 and memory 36 may each be formed of multiple separate but communicatively connected components. Software code 38 is any form of instructions or rules that direct how processing circuit 34 is to receive, interpret and respond to information to operate as described herein. Software code 38 or instructions are stored in memory 36 and accessible to processing circuit 34.

### Power Source 26:

In the arrangement shown, as one example, wearable device 12 includes a power source 26. Power source 26 is formed of any suitable size, shape, design, technology, and in any arrangement or configuration and is configured to provide power to wearable device 12 so as to facilitate the operation of the electronic circuit 24, sensors 22, and/or other electrical components of the wearable device 12. In the arrangement shown, as one example, power source 26 is formed of one or more batteries, which may or may not be rechargeable. Additionally or alternatively, in one or more arrangements, power source 26 may include a solar cell or solar panel that may power or recharge wearable device 12. Additionally or alternatively, in one or more arrangements, power source 26 may be line-power that is power that is delivered from an external power source into the wearable device 12 through a wired connection. Additionally or alternatively, in one or more arrangements, power source 26 may be a wireless power delivery system configured to power or recharge wearable device 12. Any other form of a power source 26 is hereby contemplated for use.

### Attachment Member 28:

In one or more arrangements, wearable device 12 is configured to be worn by a worker 16 and in this way, wearable device 12 is considered to be a wearable device 12. To facilitate being worn by a worker 16 while working, wearable device 12 includes an attachment member 28 connected to or formed into wearable device 12. In some various arrangements, wearable device 12 may utilize various different methods and/or means to attach with a worker 16 including but not limited to, for example, a band, strap, belt, elastic strap, snap-fit member, a clip, hook-and-loop arrangement, a button, a snap, a pin, a zipper-mechanism, a zip-tie member, a magnet, an adhesive, and/or any other attachment means, that are attachable to a worker's arm wrist, arm, ankle, leg, hand, finger, waist, chest, neck, head, or other part of the body or clothing worn by the worker 16. In one or more arrangements, it is desirable to attach the wearable device 12 to the worker's non-dominant arm while working. As another arrangement, wearable device 12 can be attached to or formed as part of a piece of clothing or equipment, such as a safety vest, a helmet or the like. In one or more arrangements, as is further described herein, wearable device 12 is held within a holster having an attachment member in a removable manner, as is further described herein.

### Wearable Devices 12 In Operation:

In some arrangements, electronic circuit 24 is configured to retrieve and evaluate data from sensors 22 to identify events of interest to facilitate selection of sensor data for analysis by monitoring system 14 and/or trigger performance of one or more actions.

For example, in one or more arrangements, electronic circuit 24 is configured to continuously monitor motion data captured by sensors 22 of wearable device 12 of a worker 16 during a work shift and evaluate the motion data to identify instances in which the motion data indicates an event of interest. In response to identifying an event of interest, a segment (or window) of the motion data in which the event occurred is communicated to the monitoring system for evaluation. Said another way, wearable device 12 pre-evaluates motion data so as to only communicate motion data when events of interest occur. Pre-evaluation of motion data by the wearable device 12 provides several benefits. Power usage by wearable device 12 for communication of data is reduced as less data is required to be transmitted to monitoring system 14. Furthermore, because less data is transmitted by wearable devices 12 more bandwidth is available for communication data and interference and collisions are reduced. Pre-evaluation of motion data by the wearable device 12 also reduces processing and storage requirements of monitoring system 14.

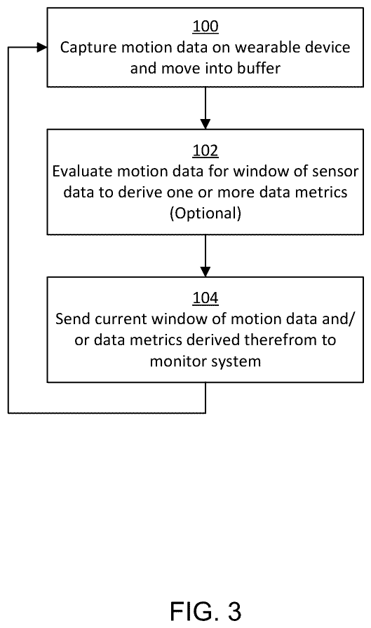

FIG. 3 shows an example process performed for collecting and processing data by a wearable device 12 in accordance with one or more arrangements. In this example, wearable device 12 operates in a continuous loop to capture motion data of a worker 16 during a work shift. At block 100, motion data is retrieved from one or more sensors 22 and placed in a buffer (or memory 36) storing a window of recent motion data (e.g., the most recent 10 seconds). At block 102, the buffered motion data is optionally processed to derive one or more data metrics from the motion data. For example in one or more arrangements at block 102 wearable device 12 may process motion data to determine the magnitude of the acceleration vector. Magnitude of the acceleration vector \vec{a} may be determined, for example, by:

$$\vert \vec{a} \vert = \sqrt{{x}^{2} + {y}^{2} + {z}^{2}}$$

At block 104, the current window of motion data and/or data metrics derived therefrom are communicated to monitor system 14. In one or more arrangements, the window of motion data includes 10 seconds of motion data. However, the arrangements are not so limited. Rather it is contemplated that in some various arrangements wearable devices may be configured to communicates motion and/or other sensor data and/or data metrics in any size windows of time.

### Communication of Data In Response to Notable Events:

While some arrangements may be primarily described with reference to wearable devices 12 continuously and/or periodically communicating sensor data and/or data metrics to monitoring system 14 during a work shift, the arrangements are not so limited. Rather, it is contemplated that in some arrangements, wearable devices 12 may be configured to communicate sensor data and/or data metrics in response to detection of notable events. For example, in some arrangements, wearable devices 12 may be configured to only communicate windows of data to monitoring system 14 in which a minimum amount of motion is detected. This may avoid unnecessary communication of motion data in which little or no motion occurs (e.g., when a worker is at rest).

Additionally or alternatively, in one or more arrangements, wearable devices may be configured to continuously and/or periodically communicate motion data and/or data metrics derived therefrom throughout a work shift as described with reference to FIG. 3 and communicate additional sensor data and/or higher density sensor data in response to detecting certain notable events (e.g., detecting a trip or a fall). Data from all sensors may be useful, for example, to facilitate analytics of the notable event by monitoring system 14.

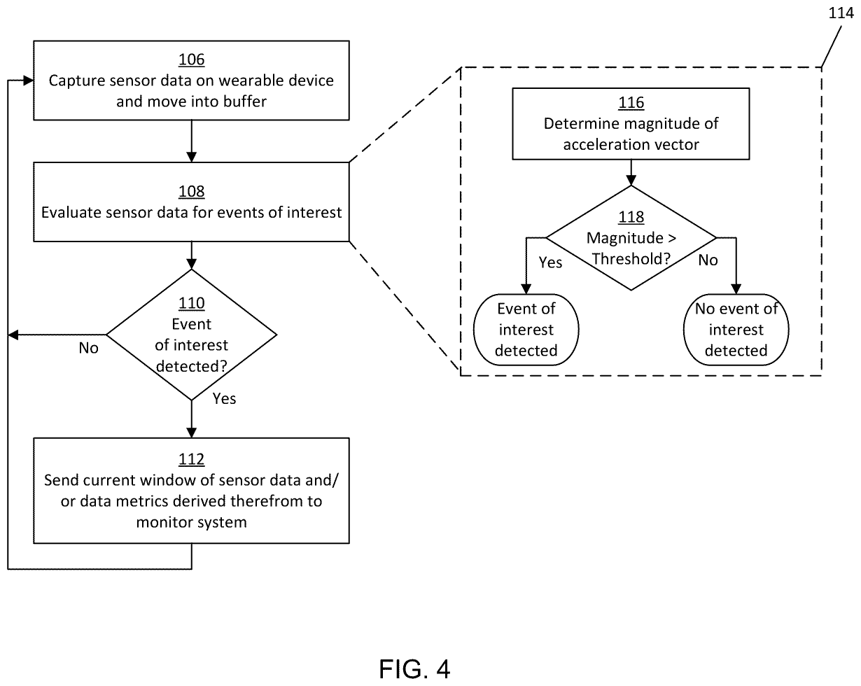

FIG. 4 shows an example process performed for collecting and processing data by a wearable device 12 in accordance with one or more arrangements. In this example, wearable device 12 operates in a continuous loop to capture sensor data of a worker 16 during a work shift. At block 106, sensor data is retrieved from one or more sensors 22 and placed in a buffer (or memory 36) storing a window of recent sensor data (e.g., the most recent 10 seconds). At block 108, the buffered sensor data is evaluated to determine if notable event is detected.

In the example shown, block 114 shown an example process for identifying events of interest. In this example, events of interest are identified when acceleration in any direction exceeds a minimal threshold. At block 116, the magnitude of the acceleration vector is determined. At decision block 118, the determined magnitude of the acceleration vector is compared to a threshold. In this example arrangement, if the movement represented by the determined magnitude exceeds that threshold an event of interest is detected. Otherwise, an event of interest is not detected.

However, the embodiments are not so limited. Rather, other thresholds may be appropriate for identifying events of interest depending on the type of activity that workers engage in during a work shift. For example in one or more arrangements, wearable devices 12 may be configured to process data acquired from motion and/or other sensors, for example using classifiers and/or other analytics processes to identify various events of interest. Such events of interest may include but are not limited to, for example, acceleration exceeding threshold, repetitive motions, specific motions or activities, excessive noise, adverse temperatures or other working conditions, worker being in close proximity to dangerous equipment, potential accidents or near misses and/or any other notable event that may be pertinent to worker safety and/or management.

If an event of interest is detected, the process proceeds from decision block 110 to block 112, where the current window of motion data and/or data metrics is communicated to monitor system 14.

If an event of interest is not detected at decision block 104, the process returns to block 100, where motion data is retrieved from one or more sensors 22 and moved into the buffer. The process repeats in this manner until wearable device 12 is powered of or operation is otherwise disabled. In one or more arrangements, wearable devices 12 are configured to sample data from sensors at approximately 25 hz. However, the embodiments are not so limited. Rather, it is contemplated that wearable devices 12 may sample data from sensors 22 at any frequency as may be appropriate for the type of data.

### Charging Base 18:

In one or more arrangements, system 10 includes a charging base 18. Charging base 18 is formed of any suitable size, shape, and design and is configured to receive, charge and transfer information from and to wearable devices 12. In the arrangement shown, as one example, charging base 18 includes a back wall 42 that includes a plurality of sockets 44 therein that are sized and shaped to receive wearable devices 12 therein. When wearable devices 12 are placed within sockets 44, wearable devices 12 are charged by charging base 18 and data may be transferred between wearable device 12 and charging base 18 and the other components of the system 10. Charging base 18 also includes a user interface 46 configured to provide the ability for the workers 16 to interact with the charging base 18. User interface 46 may include but is not limited to, for example, a plurality of sensors, a keypad, a biometric scanner, a touch screen or any other means or method input for information.

In one or more arrangements, charging base 18 is configured to facilitate checkout/checking of wearable devices 12 by workers 16. As one example, at the beginning of a shift, a worker 16 engages the charging base 18 using user interface to identify the worker with the system 10 (e.g., by biometrically scanning in with a finger or thumb print, a retinal scan, facial recognition, voice recognition, inputting a name or identifier, swiping a ID card, and/or any other manner or method of associating their personal identification with the system 10.

Upon receiving this information, charging base 18 and system 10 identifies the worker 16 and allocates a wearable device 12 held within one of the sockets 44 of the charging base 18 that is fully charged, or has the highest charge among the wearable devices 12, and assigns that wearable device 12 to that worker 16 by illuminating the wearable device 12, illuminating the socket 44 that the wearable device 12 is held in, or providing the socket number to the worker 16 or by identifying which wearable device 12 the worker 16 is to take by any other manner, method or means.

Once the proper wearable device 12 has been identified to the worker 16, the worker 16 retrieves that wearable device 12 from the charging base 18 and puts on the wearable device 12. During the work shift, the wearable device 12 gathers data from sensors 22 and communicates data to monitoring system 14 as described herein.

At the end of the shift, the worker 16 returns the wearable device 12 to the charging base 18. Once the wearable device 12 is plugged into a socket 44, the charging base 18 begins charging the wearable device 12. If the wearable device 12 has buffered data, charging base 18 retrieves the data from the wearable device 12 and provides the retrieved data to monitoring system 14.

In one or more arrangements, after turning in the wearable device 12 at the end of their shift, the worker 16 is provided with a log of all instances that were identified as events of interest. The information related to each of these potential accidents or near misses and/or notable events is provided to the worker 16 such as time, acceleration, position, temperature, light level, air quality, volume, CO level, the audible recording or converted text of the contemporaneous recording of the incident or notable event. The worker 16 is then provided the opportunity to confirm or deny whether a notable event of interest actually occurred and provide additional information regarding the notable event of interest. This provides the worker 16 the opportunity to clarify the record and provide additional information.

In one or more arrangements, the system 10 may also update the software or firmware on the wearable device 12 and prepare the wearable device 12 for another use while in the charging base. For example, in one or more arrangements, system may from time to time update classifiers or other analytics algorithms used by wearable devices 12 to identify events of interest.

### Monitoring System 14:

Monitoring system 14 is formed of any suitable size, shape, design and is configured to receive and process sensor data from wearable devices 12 to facilitate analysis of sensor data (e.g., to assess worker physicality, risk, and/or derive various other data metrics). In the arrangement shown, as one example, monitoring system 14 includes a database 60 and a data processing system 62, among other components.

### Database 60:

Database 60 is formed of any suitable size, shape, design and is configured to facilitate storage and retrieval of data. In the arrangement shown, as one example, database 60 is local data storage connected to data processing system 62 (e.g., via a data bus or electronic network 20). However, embodiments are not so limited. Rather, it is contemplated that in one or more arrangements, database 60 may be remote storage or cloud based service communicatively connected to data processing system 62 via one or more external communication networks.

In some various arrangements, information recorded by wearable devices 12 may be to database 60 for storage directly (e.g., over electronic network 20) from wearable devices. Additionally or alternatively, in some various arrangements, information recorded by wearable devices 12 may be to database 60 for storage indirectly (e.g., by charging base 18 and/or data processing system 62).

### Data Processing System 62:

Data processing system 62 is formed of any suitable size, shape, and design and is configured to facilitate receipt, storage, and/or retrieval of information in database 60, execution of analytics processes 70, providing of a user interface 72, and/or implementation of various other modules, processes or software of system 10.

In one or more arrangements, for example, such data processing system 62 includes a circuit specifically configured to carry out one or more of these or related operations/activities. For example, data processing system 62 may include discreet logic circuits or programmable logic circuits configured for implementing these operations/activities, as shown in the figures, and/or described in the specification. In certain embodiments, such a programmable circuit may include one or more programmable integrated circuits (e.g., field programmable gate arrays and/or programmable ICs). Additionally or alternatively, such a programmable circuit may include one or more processing circuits (e.g., a computer, microcontroller, system-on-chip, smart phone, server, and/or cloud computing resources). For instance, computer processing circuits may be programmed to execute a set (or sets) of instructions (and/or configuration data). The instructions (and/or configuration data) can be in the form of firmware or software stored in and accessible from a memory (circuit). Certain embodiments are directed to a computer program product (e.g., nonvolatile memory device), which includes a machine or computer-readable medium having stored thereon instructions, which may be executed by a computer (or other electronic device) to perform these operations/activities.

### User Interface 72:

User interface 72 is formed of any suitable size, shape, design, technology, and in any arrangement and is configured to facilitate user control and/or adjustment of various components of system 10. In one or more arrangements, as one example, user interface 72 includes a set of inputs (not shown). Inputs are formed of any suitable size, shape, and design and are configured to facilitate user input of data and/or control commands. In various different arrangements, inputs may include various types of controls including but not limited to, for example, buttons, switches, dials, knobs, a keyboard, a mouse, a touch pad, a touchscreen, a joystick, a roller ball, or any other form of user input. Optionally, in one or more arrangements, user interface 72 includes a display (not shown). Display is formed of any suitable size, shape, design, technology, and in any arrangement and is configured to display information of settings, sensor readings, time elapsed, and/or other information pertaining to operation and/or management of system 10. In one or more arrangements, the display may include, for example, LED lights, meters, gauges, screen or monitor of a computing device, tablet, and/or smartphone.

Additionally, or alternatively, in one or more arrangements, the inputs and/or display may be implemented on a separate device that is communicatively connected to monitoring system 14. For example, in one or more arrangements, operation of monitoring system 14 may be customized or controlled using a smartphone or other computing device that is communicatively connected to the monitoring system 14 (e.g., via Bluetooth, WIFI, and/or the internet).

### Analytics Processes 70:

In some example arrangements, data processing system 62 is configured to perform various tracking, analytics processes 70, and/or other operations described using data received from wearable devices 12 and/or data stored in database 60.

### Physicality Assessment:

In one or more arrangements, analytics processes 70 are configured to analyze sensor data received from wearable devices 12 to identify and/or predict fatigue in workers 16 that may lead to accident and/or injury. In one or more arrangements, analytics processes 70 are configured to identify and/or predict fatigue in workers 16 based on one or more data metric indicative of physicality of workers 16 during a work shift (e.g., power exerted in movements).

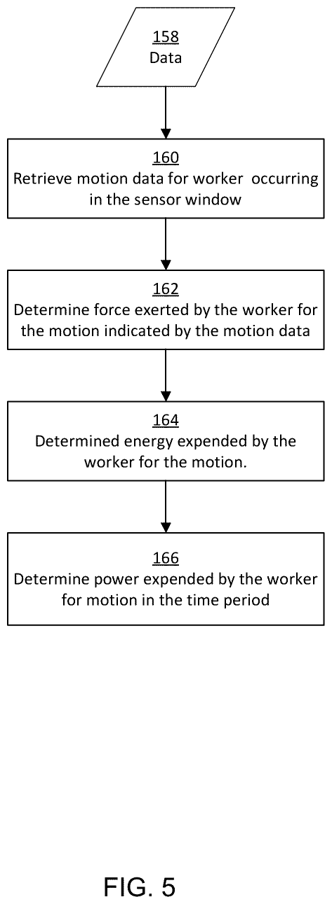

FIG. 5 shows an example dataflow arrangement for determining power exerted by a worker 16 over time, in accordance with one or more arrangements. At block 160, motion data 158 for the worker occurring in the sensor window is retrieved.

In this example, at processing block 162 force exerted by the worker in the motion is determined based on the magnitude of the acceleration vector as:

$$Force = mass * \vert \vec{a} \vert$$

In one or more arrangements, wearable devices 12 are configured to be worn on the upper arm (between the shoulder and elbow). In such arrangement, force would be calculated using the mass of the arm of the worker 16. In one or more arrangements, an estimated mass of an average arm (e.g., 4.5 kg) is used for force calculation. However, the embodiments are not so limited. Rather, it is contemplated that in some arrangements, analytics processes 70 may calculate force using a more accurate measurement of mass. For example, analytics processes 70 may calculate force using an individual mass measurement specific to each worker that is stored in database 60.

In this example, after calculating force energy is calculated at process block 164 as:

$$Energy ( J ) = Force * Distance ( meters )$$

In some arrangements, energy may be calculated using the actual distance moved in the window of sensor data (e.g. as indicated by a position sensor 22). In some arrangements, energy may be calculated using an estimated distance moved (e.g., 0.5 meters). After calculating energy, power is then calculated at process block 166 as:

$$Power = \frac{Energy}{time}$$

### Power Curves:

In one or more arrangements, analytics processes 70 are configured to identify and/or predict fatigue in workers 16 by constructing a curve of power exerted by a worker 16 during a work shift (referred to herein as a “power curve” 170). The power curve 170 indicates the determined power exerted for each window of sensor data (or calculation period).

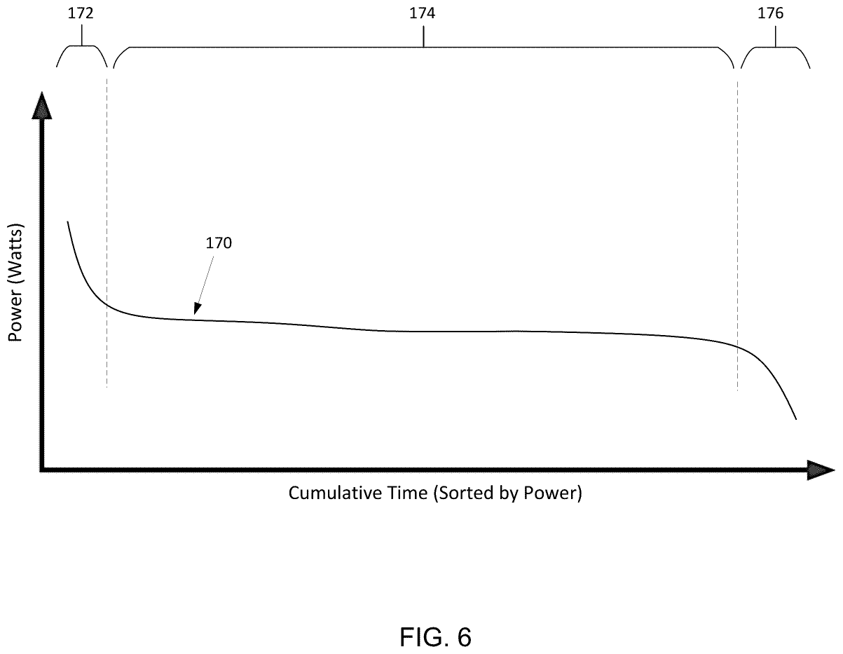

FIG. 6 shows an example power curve 170, in accordance with one or more arrangements. In this example, data values in the power curve 170 are sorted according to the level of power exerted, from highest to lowest. In this example, the left portion 172 of the power curve 170 with the highest values correlate to periods of time at which a worker 16 first begins physical exertion after resting and exhibits peak power. In such periods, the worker 16 exhibits an initial burst of strength and a high level of power which quickly fades (typically within seconds). The center plateau portion 174 of the power curve 170 represents the normal non-fatigued state of a worker, which is generally sustained for the majority of the work shift. While there may be variation in this center portion 174, the values are typically fairly consistent until workers 16 become fatigued. When workers 16 become fatigued, the power exerted rapidly dips as shown in the right portion 176 of the power curve 170. This dip represents when a worker's power output falls below the flatline and potentially suggests that they have entered a fatigued state.

### Baseline Power Curve:

In one or more arrangements, power and/or power curve 170 data of a worker 16 over a number of days is combined to generate a baseline power curve 178 (not shown). Various different arrangements nay utilize various means and/or methods to produce a baseline power curve 178. As one example, in some arrangements, a baseline power curve 178 may formed by averaging power curves 170 of the worker 16 for a set of previous work shifts (e.g., 30 or more days). Generally, the more days of power and/or power curve 170 data are included, the more accurate the baseline power curve 178 will be.

In one or more arrangements, analytics processes 70 use the baseline power curve 178 to assist in determining when a power curve 170 for a current work shift indicates when a worker 16 has and/or will become fatigued. For example, in one or more arrangements, analytics processes 70 are configured to dynamically create and update a power curve 170 for a worker 16 as motion data and/or data metrics are received from wearable device 12 of the worker 16 during the work shift. In one or more arrangements, analytics processes 70 are configured to evaluate the power curve 170 as it is created/updated to identify and/or predict when worker 16 is fatigued.

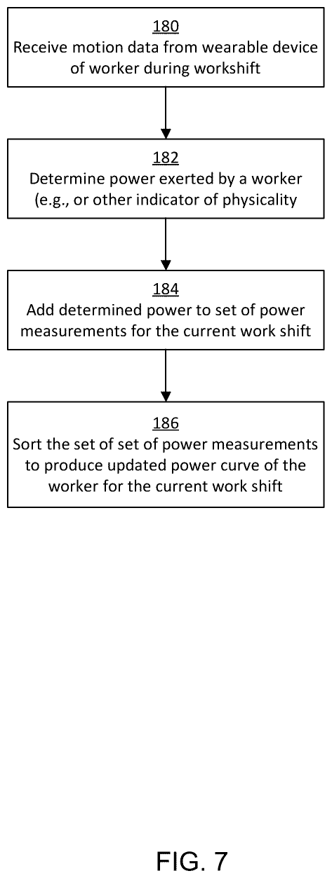

FIG. 7 shows an example process for dynamic creation and/or update of a power curve 170 during a work shift, in accordance with one or more arrangements. At block 180, motion data (and/or other data) received from wearable device 12 a time period of interest is retrieved (e.g., from database 60). At block 182, power exerted by the worker 16 (and/or other data metric(s) indicative of physicality) is derived from the retrieved data. At block 184, the process adds the determined power to a set of power measurements for the current work shift of the worker 16. At block 186, the set of power measurements for the work shift of the worker 16 is sorted to produce an updated power curve 170 for the work shift.

### Identification of Fatigue:

In one or more arrangements, after each time a power curve 170 of a worker 16 is updated, analytics processes 70 evaluate the power curve 170 to determine if the worker 16 is becoming fatigued. In some various arrangements, analytics processes 70 may utilize various methods and/or means to evaluate fatigue from the current power curve 170. In one or more arrangements, a baseline power curve 178 is evaluated to identify a lower threshold power level (e.g., threshold 190), below which a power curve 170 for the worker 16 is considered to be indicative of fatigue. The threshold level may be set, for example, based on the range of the baseline power curve 178 in the center plateau portion (e.g. portion 174 in power curve 170 of FIG. 6). For instance, in one or more arrangements, the threshold level may be set to a certain buffer amount below the minimum power level in the center portion of the baseline power curve 178. The buffer amount may help to ensure that fatigue is not prematurely detected by typical variation of power exhibited by a worker 16.

Additionally or alternatively, in one or more arrangements, analytics processes 70 may be configured to evaluate the baseline power curve 178 to identify certain trends that indicate that a worker is becoming fatigued. For instance, in some arrangement, such a trend may include, for example, the tail end of a power curve 170 having a certain downward slope (e.g., >−0.5×).

Additionally or alternatively, in one or more arrangements, analytics processes 70 may utilize artificial intelligence and/or machine learning algorithms trained, from a baseline power curve 178 and/or historical movement data, power, and/or other data metrics, to identify when a power curve 170 indicates a worker 16 is or will be fatigued. Such training may include, for example, generation and refinement of classifiers and/or state machines configured to map input data values (e.g. baseline power curves 178, motion data, and/or data metrics) fatigue levels. In various embodiments, analysis by the analytics processes 70 may include various guided and/or unguided artificial intelligence and/or machine learning techniques including, but not limited to: artificial neural networks, genetic algorithms, support vector machines, k-means, kernel regression, discriminant analysis and/or various combinations thereof. In different implementations, analysis may be performed locally, remotely, or a combination thereof.

### Prediction of Fatigue:

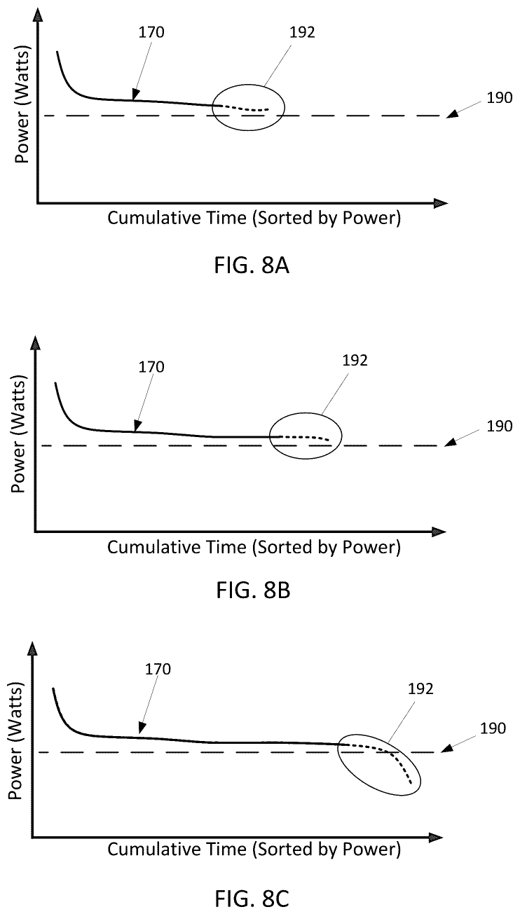

In one or more arrangements, analytics processes 70 are configured to evaluate a power curve 170 for a current work shift as it is updated to predict future values. FIGS. 8A, 8B, and 8C illustrate creation and assessment of an example power curve 170 at several points in time during a work shift for prediction of worker fatigue.

In this example, FIG. 8A shows an example power curve 170 updated at approximately 50% of the length of a work shift. In this example, a tail end of the power curve 170 is higher than the threshold 190, indicating that the power curve 170 is within the central plateau portion 174 and the worker has not yet become fatigued. Data points 192 are predicted future values generated by analytics processes 70 (e.g., based on the current power curve 170 and a baseline power curve 178). In this example, the predicted data points 192 are also above the threshold 190, indicating that the worker 16 is not predicted to become fatigued in the near future.

In this example, FIG. 8B shows power curve 170 updated at approximately 66% of the length of the work shift. In this example, a tail end of the power curve 170 remains higher than the threshold 190, indicating that the power curve 170 is within the central plateau portion 174 and the worker 16 has not yet become fatigued. In this example, the predicted data points 192 are also above the threshold 190, indicating that the worker 16 is not predicted to become fatigued in the near future.

In this example, FIG. 8C shows power curve 170 updated at approximately 80% of the length of the work shift. In this example, a tail end of the power curve 170 remains higher than the threshold 190, indicating that the power curve 170 is within the central plateau portion 174 and the worker 16 has not yet become fatigued. In this example, the predicted data points 192 dip below the threshold 190, indicating that the worker 16 is likely to become fatigued in the near future. In this manner, analytics processes 70 are able to take advanced action to mitigate risks presented by worker fatigue.

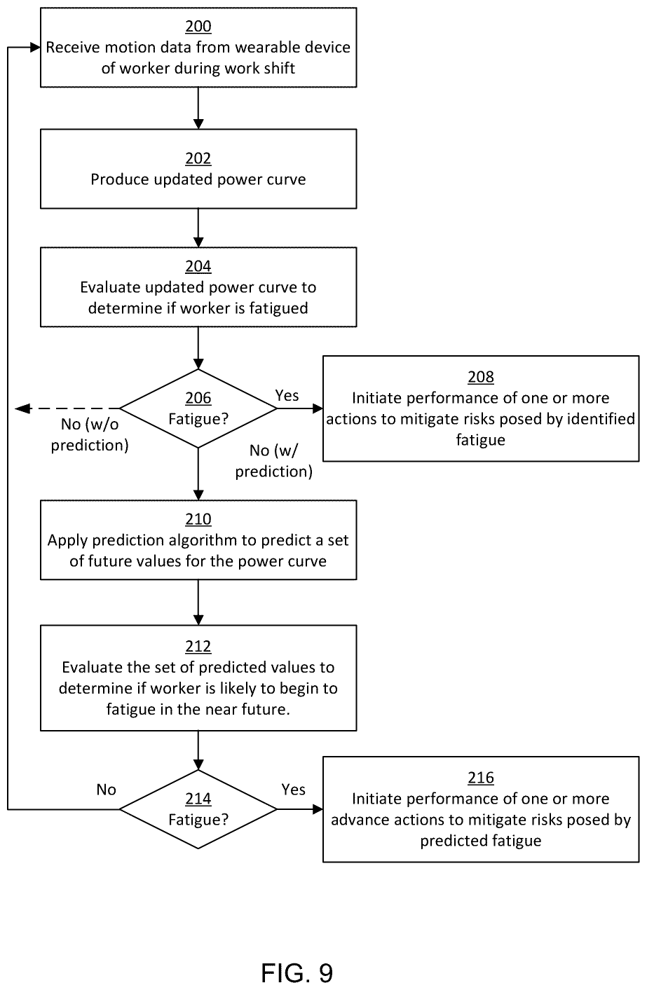

FIG. 9 shows an example process for dynamic creation and/or update of a power curve 170 and prediction of worker fatigue during a work shift, in accordance with one or more arrangements. At process block 200, motion data (and/or other data) received from wearable device 12 a time period of interest is retrieved (e.g., from database 60). At process block 202, a power curve 170 for the work shift is updated based on the received motion data (e.g., as described with reference to FIG. 7). At process block 204, the updated power curve 170 is evaluated to determine if the worker 16 has become fatigued. If the worker 16 is fatigued, the process proceeds from decision block 206 to process block 208, where monitoring system 14 initiates one or more actions are taken to mitigate risks presented by the identified fatigue. Otherwise, if the analytics processes 70 are not configured to predict fatigue, the process proceeds from decision block 206 back to process block 200 and the process is repeated.

If the analytics processes 70 are not configured to predict fatigue and fatigue is not identified in the updated power curve 170, the process proceeds from decision block 206 to process block 210. At process block 210, a prediction algorithm is applied to predict a set of future values for the power curve 170. In some various arrangements, analytics processes 70 may utilize various methods and/or means to predict future values from the current power curve 170.

For example, in some arrangements, analytics processes 70 may utilize artificial intelligence and/or machine learning algorithms trained, from a baseline power curve 178 and/or historical movement data, power, and/or other data metrics, to predict future values for the power curve 170. Such training may include, for example, generation and refinement of an artificial neural network or other machine learning algorithm to map input data values (e.g. baseline power curves 178, motion data, and/or data metrics) to fatigue levels, for example, using a gradient descent calculation to adjust the values of the artificial neural network/algorithm to minimize differences between predicted baseline power curve values and actual baseline power curve values. However, the arrangements are not so limited. Rather, it is envisioned that the analytics processes 70 may include various guided and/or unguided artificial intelligence and/or machine learning techniques including, but not limited to: artificial neural networks, genetic algorithms, support vector machines, k-means, kernel regression, discriminant analysis and/or various combinations thereof. In different implementations, analysis may be performed locally, remotely, or a combination thereof. At process block 212, the predicted values are evaluated to determine if the worker 16 is likely to begin to fatigue in the near future. If the worker 16 is likely to become fatigued in the near future, the process proceeds from decision block 214 to process block 216, where monitoring system 14 initiates one or more actions mitigate risks presented by the predicted fatigue.

### Not Limited to Power Based Assessment:

While the arrangements may be primarily described with reference to identifying/predicting fatigue from evaluation of power exhibited by workers 16, the embodiments are not so limited. Rather, it is contemplated that in one or more arrangements, fatigue assessment may additionally or alternatively be determined based on a variety of data including but not limited to, for example, motion data, work, power, heart rate, temperature, perspiration level, number of steps, distance traveled, and/or other data acquired by sensors 22 and/or derived by analytics processes 70 using data analytics (e.g., identification of repetitive motions).

Not Limited to Worker Specific Baseline Power Curves and/or Algorithms:

While some arrangements may be primarily described with reference to baseline power curves 178 that are created specific to individual workers 16, the arrangements are not so limited. Rather, it is contemplated that baseline power curves 178 may additionally or alternatively be more generically created for groups of workers (e.g., of the same job type), for specific work assignments or types of work, or any other classification of workers 16 or work assignments.

Similarly, it is contemplated that utilize artificial intelligence and/or machine learning algorithms (e.g., used for evaluation of fatigue and/or power curve prediction) may be trained for individual workers, for groups of workers, or for specific work assignments or types of work, or any other classification of workers 16 or work assignments.

In some arrangements, analytics processes 70 may be configured to utilize multiple different baseline power curves and/or multiple artificial intelligence and/or machine learning algorithms when evaluation of fatigue and/or predicting power curve data for a particular worker. For example, in some arrangements, fatigue evaluation and/or power curve data prediction may include using a combination of worker specific, group specific and/or work specific baseline power curves 178 and/or artificial intelligence and/or machine learning algorithms. For instance, in one or more arrangements, analytics processes 70 may be configured to perform respective power curve prediction assessments of a worker 16 using worker specific, group specific and/or work specific baseline power curve 178 and/or algorithms and combine the results (e.g., by averaging).

### Management Software 74:

In one or more arrangements, information provided by wearable devices 12 and/or derived by analytics processes 70 is processed by management software 74.

In some arrangements, management software 74 may be configured to perform various actions based on fatigue status of workers 16. For example, in one or more arrangements, management software 74 may be configured to generate reports, send alerts to workers 16 and/or managers (e.g., text message, IM. email, automated call, and/or other means for communication), and/or initiate one or more mitigative actions in response to analytics processes 70 determining or predicting that a worker 16 is fatigued or will become fatigued in the near future.

### Example Mitigative Actions:

In some various arrangements, management software 74 is configured to automatically initiate various different actions to mitigate risks presented by the identified and/or predicted fatigue of workers 16. In some various arrangements, management software 74 realizes an improvement in operations and safety by avoiding the delay involved in waiting for an administrator to react to detected/predicted fatigue by automatically performing such actions.

### Status Boards and Alerts:

As an illustrative example, some worksites have status boards 220 (not shown) located nearby production lines and/or workstations identifying workers 16 currently working on the production line and/or workstation. The status board 220 may also show additional information for each worker 16 such as time started, length of time at the production line and/or workstation, time scheduled for shift rotation, and/or any other relevant information.

As a mitigative measure, in one or more arrangements, monitoring system 14 may be configured to cause the status board 220 to provide one or more visual indicators
(e.g., color, warning icon, flashing, etc.) to indicate when workers 16 are identified as being fatigued or predicted to become fatigued in the near future. In some arrangement, monitoring system 14 may be configured to dynamically quantify a fatigue level for each worker during the work shift and cause status board 220 to display a visual indicator of the fatigue level for each worker (e.g., text, percentage, gauge, life meter, etc.). Additionally or alternatively, in one or more arrangements, monitoring system 14 may be configured to initiate audible, haptic, electronic messaging alerts (e.g., email, SMS, IM, and/or any other electronic messaging) in response to identifying that a worker 16 is or will likely become fatigued in the near future.

In this manner workers and/or managers can quickly assess status of workers 16 and rotate workers off of dangerous work assignment when identified as fatigued or predicted to become fatigued in the near future.

Restricting Permitted Access and/or Operation for Fatigued Workers:

As an example mitigative measure, in one or more arrangements, monitoring system 14 may be configured to automatically control operation of various other production equipment and/or devices based on the fatigue status of workers 16 to mitigate risk of accident and/or injury. For example, in one or more arrangements, monitoring system 14 may be configured to disable dangerous machinery and/or adjust permissions of a workers, for example, to prevent a worker 16 that has been identified as or predicted to soon be fatigued from access/using dangerous machinery and/or locations.

For instance, in one or more arrangements, monitoring system 14 may be configured to generate control signals and/or control switching of one or more relay switches (not shown) in response to determined worker statuses (fatigue, locations, etc.) and/or based on other sensor data gathered by wearable devices 12. Such control signals and/or relay switches may be configured to control operation of various devices (e.g., lights, alarms, locks, doors, equipment, and/or any other devices) to mitigate risks posed by fatigued workers 16.

As an illustrative example, in one or more arrangements, monitoring system 14 is configured to control a relay switch connected to a door lock to control access to an area that is restricted due to high risk of injury. To enable access, a worker 16 may hold their wearable device 12 near a nearby sensor to identify the worker 16 to the system 10. Once identified, monitoring system 14 may check permissions of the worker 16 to verify that the worker is permitted access. If access is permitted, the monitoring system 14 may then cause relay switch to unlock the door lock. However, in one or more arrangements, the monitoring system 14 may be configured to deny access to a worker 16 normally permitted access to the restricted area if the worker 16 is determined to be or predicted to soon be fatigued. In this manner, fatigued worker can be kept away from potentially dangerous areas while fatigued.

As another illustrative example, in one or more arrangements, monitoring system 14 is configured to enable/disable operation of certain dangerous equipment (e.g., forklift, saw, lathe, etc.) so as to only permit authorized workers 16 to operate such equipment.

To enable operation, a worker 16 may hold their wearable device 12 near a nearby sensor to identify the worker 16 to the system 10. Once identified, monitoring system 14 may check permissions of the worker 16 to verify that the worker is authorized to operate the equipment. If operation is permitted, the monitoring system 14 may then prompt a cause relay switch (or other control circuit) to enable operation of the equipment. However, in one or more arrangements, the monitoring system 14 may be configured to keep the equipment disabled for the worker 16 normally authorized to operate if the worker 16 is determined to be or predicted to soon be fatigued. In this manner, fatigued worker can be prevented from operating potentially dangerous equipment while fatigued.

### Automated Scheduling of Breaks for Workers:

As yet an example mitigative measure, in one or more arrangements, monitoring system 14 may be configured to automatically prompt a worker 16 to take breaks, switch to a less demanding work assignment, and/or move to a more comfortable area in response to determining the worker 16 is fatigued or will become fatigued in the near future. In some arrangements, monitoring system 14 may prompt the worker 16 directly, for example by sending a message or alert (e.g., via visual alert, audible alert, email, SMS, IM, or other electronic messaging) to the worker 16, or prompt the worker 16 indirectly, for example, by sending a message or alert to a manager. However, the arrangements are not so limited. Rather, it is contemplated that in various arrangements, monitoring system 14 may be configured to automatically prompt a worker 16 to take breaks, switch to a less demanding work assignment, and/or move to a more comfortable area using any known method and/or means.

### Not Limited to the Example Mitigating Actions:

It should be recognized that the arrangements are limited to these example actions to mitigate risk. Rather, it is contemplated that in some various arrangements, monitoring system 14 may be configured to (or customized by an authorized user for the work site to) automatically perform any action in response to identifying a worker as being fatigued and/or predicting a worker 16 will become fatigued in the near future.

From the above discussion, it will be appreciated that one or more arrangements provide a wearable device, system, and/or method of use presented improves upon the state of the art. Specifically, one or more arrangements provide a wearable device, system, and/or method: for collecting, reporting and analyzing information indicative of work performed by workers and/or conditions that workers are exposed to in a workplace to better assess physicality of workers and worker fatigue; that identifies when workers have become fatigued based on the physicality; that predicts when workers will become fatigued based on the physicality; that identifies and/or predicts when workers are fatigued and automatically performs a set of actions to mitigate risk of injury when fatigue is identified and/or predicted; that analyzes data gathered to assess worker fatigue at multiple times throughout a work shift; that more accurately assesses risk during a work shift; that assesses gathered data indicative of work performed by workers and workplace conditions to facilitate assessment of safety risks faced by workers during a work shift; that aggregates a great amount of information indicative of work performed by workers and workplace conditions to facilitate data analytics; that is cost effective; that is safe to use; that is easy to use; that is efficient to use; that is durable; that is robust; that has a long useful life; that can be used with a wide variety of occupations; that provides high quality data; and/or that provides data and information that can be relied upon.

These and countless other objects, features, or advantages of the present disclosure will become apparent from the specification, figures, and claims.

### SELECTED REFERENCE NUMBERS

10—System
12—Wearable Devices
14—Monitoring System
16—Worker
18—Charging Base
20—Network
22—Sensors
24—Electronic Circuit
26—Power source
28—Attachment Member
32—Communication Circuit (of Electronic Circuit 24)
34—Processing Circuit (of Electronic Circuit 24)
36—Memory
38—Software Code
42—Back Wall (of Charging Base 18)
44—Sockets (of Charging Base 18)
46—User Interface (of Charging Base 18)
60—Database
62—Data Processing System
70—Analytics Processes
72—User Interface
74—Management Software
100—Block
102—Block
104—Block
106—Block
108—Block
110—Decision Block
112—Block
114—Process
116—Block
118—Decision Block
130—Block
132—Block
134—Decision Block
136—Block
138—Decision Block
140—Block
142—Decision Block
144—Block
150—Block
152—Block
154—Block
158—Motion Data
160—Block
162—Block
164—Block
166—Block
168—Block
170—Power Curve
172—Left Portion (of Power Curve)
174—Center Portion (of Power Curve)
176—Right Portion (of Power Curve)
178—Baseline Power Curve
180—Block
182—Block
184—Block
186—Block
190—Threshold
192—Predicted Values
200—Block
202—Block
204—Block
206—Decision Block
208—Block
210—Block
212—Block
214—Decision Block
216—Block
220—Status Board

What is claimed:

1. A system for evaluating worker safety, comprising;
a wearable device;
a monitoring system;
the wearable device communicatively connected to the monitoring system;
wherein the wearable device is configured to be worn by a worker during a work shift;
wherein the wearable device includes one or more sensors;
wherein the one or more sensors includes a motion sensor;
wherein the wearable device is configured to communicate windows of motion data recorded by the motion sensor to the monitoring system;
wherein the monitoring system is configured to perform analytics on the motion data to assess fatigue of the worker.

2. The system of claim 1, wherein the analytics performed by the monitoring system is configured to assess fatigue of the worker by:
determining power levels exerted by the worker from the motion data over time; and
identifying when the worker is becoming fatigued based on the determined power levels exerted by the worker.

3. The system of claim 1, wherein the analytics performed by the monitoring system is configured to assess fatigue of the worker by:
determining power levels exerted by the worker from the motion data;
creating a power curve from the determined power levels; and
identifying when the worker is becoming fatigued based on the power curve.

4. The system of claim 1, wherein the analytics performed by the monitoring system is configured to assess fatigue of the worker by:
determining a power level exerted by the worker in each window of motion data;
sorting the determined power levels from highest to lowest to create a power curve; and
identifying when the worker is becoming fatigued based on the power curve.

5. The system of claim 1, wherein the analytics performed by the monitoring system is configured to assess fatigue of the worker by:
determining a power level exerted by the worker in each window of motion data;
sorting the determined power levels from highest to lowest to create a power curve; and
identifying when the worker is becoming fatigued based on a comparison of the power curve to a baseline power curve for the worker.

6. The system of claim 1, wherein the analytics performed by the monitoring system is configured to assess fatigue of the worker by:
determining a power level exerted by the worker in each window of motion data;
sorting the determined power levels from highest to lowest to create a power curve; and
identifying when the worker is becoming fatigued based on a comparison of the power curve to a threshold power level for the worker.

7. The system of claim 1, wherein the analytics performed by the monitoring system is configured to assess fatigue of the worker by:
determining a power level exerted by the worker in each window of motion data;
sorting the determined power levels from highest to lowest to create a power curve; and
identifying when the worker is becoming fatigued by assessing the power curve using a machine learning algorithm that is trained to identify from the power curve when the worker is becoming fatigued.

8. The system of claim 1, wherein the analytics performed by the monitoring system is configured to assess fatigue of the worker by, for each window of motion data received during the work shift:
determining a power level exerted by the worker in the window of the motion data;
adding the determined power level to a set of power measurements;
sorting the set of power measurements to create a power curve; and
identifying when the worker is becoming fatigued based on the power curve.

9. The system of claim 1, wherein the analytics performed by the monitoring system is configured to assess fatigue of the worker by, for each window of motion data received during the work shift:
determining a power level exerted by the worker in the window of the motion data;
adding the determined power level to a set of power measurements;
sorting the set of power measurements to create a power curve; and
performing analytics on the power curve to predict a set of future power measurements for the power curve;
determining if the worker will become fatigued in the near future based on the set of future power measurements.

10. The system of claim 1, wherein the analytics performed by the monitoring system is configured to assess fatigue of the worker by, for each window of the motion data received during the work shift:
determining a power level exerted by the worker in the window of the motion data;
adding the determined power level to a set of power measurements;
sorting the set of power measurements to create a power curve; and
performing analytics on the power curve to predict a set of future power measurements for the power curve;
determining if the worker will become fatigued in the near future based on a comparison of the set of future power measurements to a baseline power curve.

11. The system of claim 1, wherein in response to identifying that the worker is becoming fatigued, the monitoring system is configured to initiate one or more actions to mitigate the risk of accident or injury due to fatigue of the worker.

12. The system of claim 1, further comprising a status board;
the status board configured to display workers currently working at a workstation;
wherein in response to determining that one of the workers currently working at the workstation is becoming fatigued, the monitoring system is configured to cause the status board to display a visual indicator warning that the worker is becoming fatigued.

13. The system of claim 1, further comprising a status board;
the status board configured to display workers currently working at a workstation;
wherein the status board is configured to receive determined fatigue levels from the monitoring system for the workers currently working at the workstation;
wherein the status board is configured to display the determined fatigue levels for the workers currently working at the workstation.

14. The system of claim 1, wherein in response to identifying that the worker is becoming fatigued, the monitoring system is configured to communicate a prompt for the worker to take a break.

15. The system of claim 1, wherein in response to identifying that the worker is becoming fatigued, the monitoring system is configured to communicate a prompt for the worker to switch to a different work assignment.

16. The system of claim 1, wherein in response to identifying that the worker is becoming fatigued, the monitoring system is configured to communicate a prompt for the worker to relocate to a more comfortable work location.

17. The system of claim 1, wherein in response to identifying that the worker is becoming fatigued, the monitoring system is configured to prevent the workers from being permitted access to one or more restricted areas.

18. The system of claim 1, wherein in response to identifying that the worker is becoming fatigued, the monitoring system is configured to prevent the workers from being permitted to operate one or more pieces of dangerous equipment.

19. A system for evaluating worker safety, comprising:
a wearable device;
a monitoring system;
the wearable device communicatively connected to the monitoring system;
wherein the wearable device is configured to be worn by a worker during a work shift;
wherein the wearable device includes one or more sensors;
wherein the one or more sensors includes a motion sensor;
wherein the wearable device is configured to communicate windows of motion data recorded by the motion sensor to the monitoring system;
wherein the monitoring system is configured to determine a power level exerted by the worker in each window of the motion data;
wherein the monitoring system is configured to sort the determined power levels from highest to lowest to create a power curve; and
wherein the monitoring system is configured to assess fatigue of the worker based on the power curve.

20. The system of claim 19, wherein the monitoring system is configured to identify when the worker has become fatigued based on the power curve.

21. The system of claim 19, wherein the monitoring system is configured to identify when the worker will become fatigued in the near future based on the power curve.

22. The system of claim 19, wherein the monitoring system is configured to identify when the worker is becoming fatigued based on a comparison of the power curve to a baseline power curve for the worker.

23. The system of claim 19, wherein the monitoring system is configured to identify when the worker is becoming fatigued by assessing the power curve using a machine learning algorithm that is trained to identify from the power curve when the worker is becoming fatigued.

24. The system of claim 19, further comprising a status board;
the status board configured to display an indicator representative of the worker as they work at a workstation;
wherein in response to determine that the worker while working at the workstation is becoming fatigued, the monitoring system is configured to cause the status board to display a visual indicator warning that the worker is becoming fatigued.

25. The system of claim 19, further comprising a status board;
the status board configured to display an indicator representative of the worker as they work at a workstation;
wherein the status board is configured to receive determined fatigue levels from the monitoring system for the worker while the worker is working at the workstation;
wherein the status board is configured to display the determined fatigue levels for the worker as they work at the workstation.

26. The system of claim 19, wherein in response to identifying that the worker is becoming fatigued, the monitoring system is configured to communicate a prompt for the worker to take a break.

27. The system of claim 19, wherein in response to identifying that the worker is becoming fatigued, the monitoring system is configured to communicate a prompt for the worker to switch to a different work assignment.

28. The system of claim 19, wherein in response to identifying that a worker is becoming fatigued, the monitoring system is configured to communicate a prompt for the worker to relocate to a more comfortable work location.

29. The system of claim 19, wherein in response to identifying that a worker is becoming fatigued, the monitoring system is configured to prevent the workers from being permitted access to one or more restricted areas.

30. The system of claim 19, wherein in response to identifying that a worker is becoming fatigued, the monitoring system is configured to prevent the workers from being permitted to operate one or more pieces of dangerous equipment.
